(19) United States
(12) Patent Application Publication
(10) Pub. No.: US 2012/0245409 A1
(43) Pub. Date: Sep. 27, 2012

(54) DEVICE FOR CONVERTING MUSIC SIGNAL TO ELECTRICAL STIMULATION

(75) Inventor: CHIH-KUO LIANG, TAINAN CITY (TW)

(73) Assignee: SOUTHERN TAIWAN UNIVERSITY OF TECHNOLOGY, TAINAN CITY (TW)

(21) Appl. No.: 13/423,380

(22) Filed: Mar. 19, 2012

(30) Foreign Application Priority Data
Mar. 21, 2011 (TW) 100109510

(51) Int. Cl. A61N 1/36; A61M 21/02

(52) U.S. Cl. 600/27; 600/26

(57) ABSTRACT

A device for converting music signals into electrical stimulation is revealed. The device for converting music signals into electrical stimulation includes a switching device. The switching device is disposed with an amplifier whose input end is connected to a music player while an output end of the amplifier is connected to a voltage/current converter. A stimulating electrode is at an output end of the voltage/current converter. Music signals are input through the music player, amplified by the amplifier, and converted by the voltage/current converter. Thus the stimulating electrode at the output end stimulates users' acupuncture points for treatment of insomnia. Thereby the treatment of insomnia is effective and the sleep quality is improved. The switching device is with a high practical value.

### BACKGROUND OF THE INVENTION

1. Field of the Invention

The present invention relates to a device for converting music signals into electrical stimulation, especially to a switching device that provides an effective treatment of insomnia and improves sleep quality by music and stimulation of acupuncture points.

2. Description of Related Art

We spend nearly about one-third of our lives. Sleep quality is the most important factor associated with our health. With the transformation of the society, many people including the young and the elderly have sleep problems. Music therapy is one of sleeping aids available now. Music influences our behavior by affecting the brain and subsequently our bodies and music therapy is a technique that uses music within a therapeutic relationship to address physical, emotional, cognitive, and social needs of individuals. Once the music is combined with brainwave signals, people's brain waves are affected by the music and going to have the same rhythm. Thus they feel calm and relaxed.

Moreover, transcutaneous electrical nerve stimulator (TENS) is widely used for pain management in rehabilitation. TENS is a kind of treatment using low-frequency electrical current applied through the skin to relieve or treat pain and is one of non-pharmacological options in general guidelines for managing pain developed by WHO (world health organization). In some research, TENS is combined with electro-acupuncture (EA) to form Acupuncture-Like Electrical Stimulation (AL-TENS). The AL-TENS using non-invasive electrode pad in treatment of insomnia also achieves similar treatment results.

Although sleep aids available on the market help relieve symptoms of insomnia, it is found that the sleep aid or the treatment method deals with insomnia only by a single means. They provide a limited effect on the treatment of insomnia.

Thus there is room for improvement and a need to provide a device for converting music signals into electrical stimulation with a high practical value.

### SUMMARY OF THE INVENTION

Therefore it is a primary object of the present invention to provide a device for converting music signals into electrical stimulation that treats sleep disorders and improves sleep quality by music and stimulation of acupuncture points.

In order to achieve the above object, a device for converting music signals into electrical stimulation includes a switching device with an amplifier. An input end of the amplifier is connected to a music player while an output end of the amplifier is connected with a voltage/current converter. An output end of the voltage/current converter is joined with a stimulating electrode. Through the music player, music signals are input into the switching device to be amplified by the amplifier and converted by the voltage/current converter. Thus the stimulating electrode at the output end stimulates users' acupuncture points for treatment of insomnia.

### BRIEF DESCRIPTION OF THE DRAWINGS

The structure and the technical means adopted by the present invention to achieve the above and other objects can be best understood by referring to the following detailed description of the preferred embodiments and the accompanying drawings, wherein:

FIG. 1 is a block diagram of an embodiment according to the present invention;

FIG. 2 is a block diagram of another embodiment according to the present invention.

### DETAILED DESCRIPTION OF THE PREFERRED EMBODIMENT

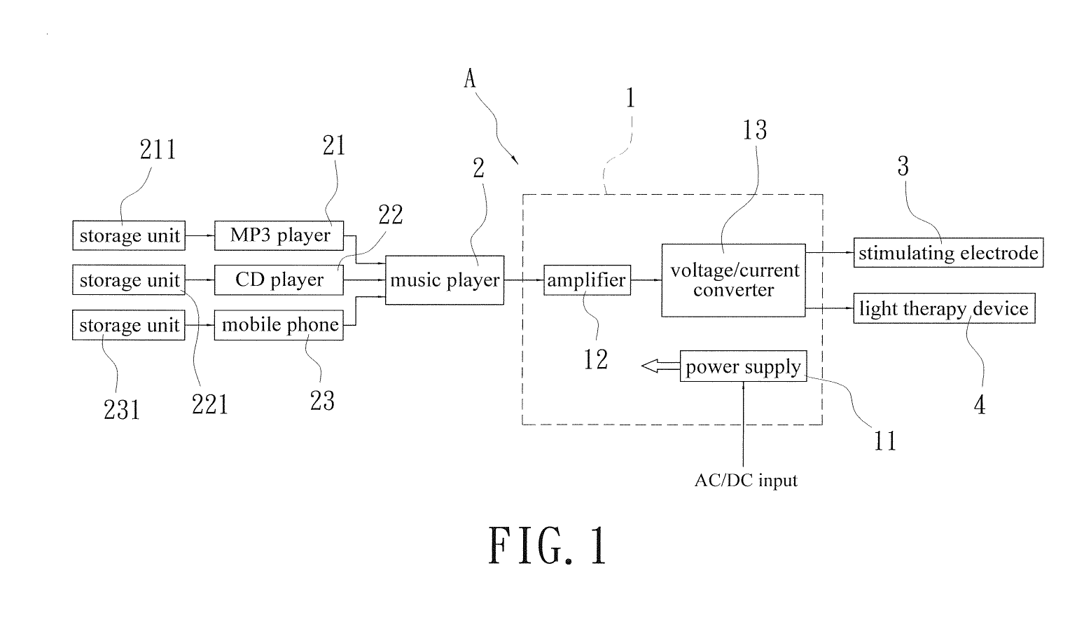

Refer to FIG. 1, a device for converting music signals into electrical stimulation (A) of the present invention includes a switching device 1, a music player 2, a stimulating electrode 3 and a light therapy device 4.

The switching device 1 is arranged with a power supply 11 while the power supply 11 is connected to a city power or at least one battery for AC(alternating current)/DC(direct current) input. The voltage input is processed by the power supply 11 and then is transmitted to be used by the switching device 1. The switching device 1 is disposed with an amplifier 12 whose input end is connected to the music player 2. The music player 2 can be a MPEG(Motion Pictures Expert Group)-1 Audio Layer-3 player 21, a CD (compact disc) player 22 or a mobile phone 23. These players are able to be input with audio signals or therapeutic signals stored in respective storage unit 211, 221, 231 such as a memory card. The therapeutic signals are signals of emotional release or electroencephalographic signals. An output end of the amplifier 12 is connected to a voltage/current converter 13 and an output end of the voltage/current converter 13 is connected to both the stimulating electrode 3 and the light therapy device 4.

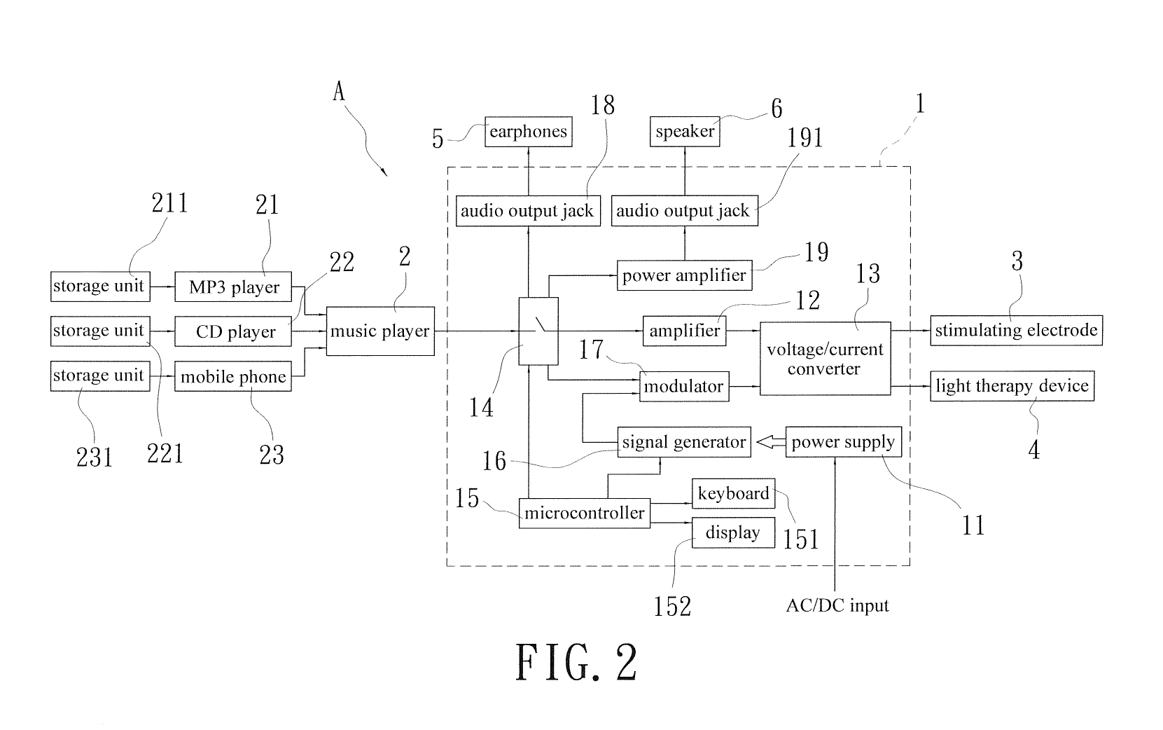

Refer to FIG. 2, another embodiment of the present invention is revealed. In this embodiment, a switch 14 is arranged between the amplifier 12 and the music player 2. The switch 14 is connected to and is controlled by a microcontroller 15. The microcontroller 15 is connected to a keyboard 151 and a display 152. The keyboard 151 is used to give control commands to the microcontroller 15 while the display 152 is to show various types of information. The microcontroller 15 is further connected to a signal generator 16 whose output end is joined with a modulator 17. The modulator 17 is also connected to the switch 14 and an output end of the modulator 17 is connected to the voltage/current converter 13. The switch 14 is further connected to an audio output jack 18 and a power amplifier 19. A pair of earphones 5 is plugged into the audio output jack 18 while the power amplifier 19 is also connected to an audio output jack 191 which a speaker 6 is plugged into.

While in use, the music player 2 is connected to the input end of the switching device 1. Through the music player 2, music signals are input into the switching device 1, amplified by the amplifier 12 and then converted by the voltage/current converter 13. Thus the stimulating electrode 3 or the light therapy device 4 connected to the output end of the switching device 1 can simulate users' acupuncture points or perform light therapy for treatment of insomnia. The keyboard 151 is used to input control commands to the microcontroller 15 and the operation state is shown on the display 152. The switch 14 is controlled by the microcontroller 15 to change to different operation status. Thus users can listen to the music by plugging the earphones 5 into the audio output jack 18. The music is input through the music player 2. Or the operation status is switched to another one that the music signals input through the music player 2 are amplified by the power amplifier 19 and then to be played by the speaker 6 connected to the audio output jack 191. Or the music signals input through the music player 2 are amplified by the amplifier 12 and then are converted by the voltage/current converter 13 to make the stimulating electrode 3 stimulate acupuncture points for treatment of insomnia or make the light therapy device 4 work for treatment of sleep disorders. Or different output waveforms generated by the signal generator 16 under the control of the microcontroller 15 together with the music signals from the music player 2 are modulated by the modulator 17 and then are converted by the voltage/current converter 13 so that the simulating electrode 3 or the light therapy device 4 at the output end can stimulate acupuncture points or take light therapy for treatment and healing of insomnia. Or select therapeutic signals containing signals of emotional release and electroencephalographic signals and stored in the respective storage unit 211, 221, 231. Firstly, select signals of emotional release or electroencephalographic signals that are going to be executed. Then the signals selected are amplified by the amplifier 12 and converted by the voltage/current converter 13 so as to make the simulating electrode 3 connected to the output end of the switching device stimulate acupuncture points or make the light therapy device 4 connected to the output end of the switching device work for treatment and healing of insomnia. Thus there are multiple operation statuses to be selected for health care and treatment of insomnia.

In summary, compared with conventional device, the present invention converts the music signals input through the music player into electrical stimulation to stimulate users' acupuncture points for treatment of insomnia. The signals of emotional release or electroencephalographic signals are converted by the switching device of the present invention to make the stimulating electrode stimulate users' acupuncture points or make the light therapy device work for effective treatment of sleep disorders and improvement of sleep quality. Thus the switching device of the present invention is more practical while in use.

Additional advantages and modifications will readily occur to those skilled in the art. Therefore, the invention in its broader aspects is not limited to the specific details, and representative devices shown and described herein. Accordingly, various modifications may be made without departing from the spirit or scope of the general inventive concept as defined by the appended claims and their equivalent.

What is claimed is:

1. A device for converting music signals into electrical stimulation comprising: a switching device; wherein the switching device is disposed with an amplifier; an input end of the amplifier is connected to at least one music player while an output end of the amplifier is connected to a voltage/current converter; an output end of the voltage/current converter is connected to at least one stimulating electrode.

2. The device as claimed in claim 1, wherein the music player is a MPEG (Motion Pictures Expert Group)-1 Audio Layer-3 player.

3. The device as claimed in claim 1, wherein the music player is a compact disc (CD) player.

4. The device as claimed in claim 1, wherein the music player is a mobile phone.

5. The device as claimed in claim 1, wherein at least one audio signal is input into the music player from at least one storage unit that stores audio signals.

6. The device as claimed in claim 1, wherein at least one signal is input into the music player from at least one storage unit that stores therapeutic signals.

7. The device as claimed in claim 6, wherein the therapeutic signals are signals of emotional release.

8. The device as claimed in claim 6, wherein the therapeutic signals are electroencephalographic signals.

9. The device as claimed in claim 1, wherein an output end of the voltage/current converter is connected to at least one light therapy device.

10. The device as claimed in claim 1, wherein the switching device is disposed with a power supply.

11. The device as claimed in claim 1, wherein a switch is arranged between the amplifier and the music player; the switch is connected to a microcontroller and the microcontroller is connected to a signal generator; an output end of the signal generator is joined with a modulator that is connected to the switch while an output end of the modulator is connected to the voltage/current converter.

12. The device as claimed in claim 11, wherein the microcontroller is connected to a keyboard and a display.

13. The device as claimed in claim 11, wherein the switch is connected to at least one audio output jack which a pair of earphones is plugged in.

14. The device as claimed in claim 11, wherein the switch is connected to a power amplifier while the power amplifier is connected to at least one audio output jack which a speaker is plugged in.
